(19) United States
(12) Patent Application Publication
(10) Pub. No.: US 2023/0197206 A1
(43) Pub. Date: Jun. 22, 2023

(54) METHOD AND DEVICE FOR ANALYZING SIALIC-ACID-CONTAINING GLYCAN

(71) Applicant: SHIMADZU CORPORTION, Kyoto-shi (JP)

(72) Inventor: Masaki MURASE, Kyoto-shi (JP)

(73) Assignee: SHIMADZU CORPORTION, Kyoto-shi (JP)

(21) Appl. No.: 18/080,009

(22) Filed: Dec. 13, 2022

(30) Foreign Application Priority Data
Dec. 16, 2021 (JP) 2021-204044

(51) Int. Cl. G16C 20/20

(52) U.S. Cl. CPC G16C 20/20

(57) ABSTRACT

Provided is a method for analyzing a sample containing a sialic-acid-containing glycan including a modification specific to a sialic-acid linkage type. A representative peak is detected from mass spectrum data of a sample for each isotope peak cluster. For each representative peak, a glycan composition is estimated according to a specified glycan search condition, and a glycan-composition candidate is determined. From the representative peaks, an isomer peak cluster including peaks estimated to be identical in the number of sialic acids and the glycan composition exclusive of the sialic acids is detected. The candidates are narrowed down by applying, to each peak in the isomer peak cluster, predetermined constraint conditions concerning the number of sialic acids contained, number of sialic-acid linkage types, kinds of sialic acids, and identity of the glycan composition exclusive of the sialic acids. A candidate list, with visual discrimination between the selected and unselected candidates, is displayed.

### TECHNICAL FIELD

The present invention relates to a method and device for analyzing a glycan by means of mass spectrometry, and more specifically, to an analysis method and analyzing device capable of an analysis of a sialic-acid-containing glycan, including the glycosidic linkage type of sialic acids. The term “glycan” in the present description includes not only a glycan in its independent form but also a form of a glycosylation, i.e., a glycan modifying a protein, peptide, lipid, nucleic acid or other kinds of biomolecules.

### BACKGROUND ART

An analysis of glycans is a major theme in bioscience, drug discovery, medicinal treatment and other related areas. Among other things, understanding the linkage type of sialic acids in a glycan which contains sialic acids is an important task in a glycan structure analysis. With such a technical background, techniques for a chemical modification specific to the linkage type of sialic acids have been developed in order to enable an efficient structural analysis of sialic-acid-containing glycans, including their difference in linkage type, by means of mass spectrometry. For example, Patent Literature 1 and Non Patent Literature 1 disclose a method for a sialic-acid-linkage specific modification, named SALSA (Sialic Acid Linkage-Specific Alkylamidation).

SALSA is a method which achieves isopropyl amidation and methyl amidation of α2,3- and α2,6-sialic acids, respectively, by utilizing the fact that the carboxylic acid in each of those sialic acids reacts in a different way when reacting with an amine to form an amide. This derivatization causes a mass difference of 28 Da between the α2,3- and α2,6-sialic acids, making it possible to distinguish between the α2,3- and α2,6-sialic acids based on the result of a mass spectrometric analysis.

Patent Literature 1 discloses a method for analyzing a glycan based on mass spectrum data obtained by a mass spectrometric analysis in which the SALSA method is employed as a pretreatment method. In the analysis method described in the document, three peaks located at intervals of 28 Da in a mass spectrum are detected as sialic-acid-linkage isomer ion peaks originating from sialic-acid-containing glycans, and an exhaustive search for compositions, with the kinds and numbers of monosaccharides as the search conditions, is performed for those peaks to estimate the glycan composition. From among the glycan-composition candidates obtained by the estimation, a composition which includes two or more α2,6-linkages is extracted as a highly plausible composition candidate for a peak which contains two or more sialic acids and shows the largest mass. The result can be displayed in the form of a table in which that highly plausible composition candidate is visually discriminated from the other composition candidates which are rather implausible to be sialic-acid-containing glycan isomers (for example, see FIGS. 6 and 8 in Patent Literature 1).

### CITATION LIST

### Patent Literature

Patent Literature 1: WO 2017/145496 A

### Non Patent Literature

Non Patent Literature 1: Takashi Nishikaze and five other authors, “Differentiation of Sialyl Linkage Isomers by One-Pot Sialic Acid Derivatization for Mass Spectrometry-Based Glycan Profiling”, Analytical Chemistry, 2017, Vol. 89, pp. 2353-2360

### SUMMARY OF INVENTION

### Technical Problem

The analysis method described in Patent Literature 1 is a technique for a structural analysis of glycans containing one specific kind of sialic acid, such as N-acetylneuraminic acid (Neu5Ac). There are a plurality of known molecular species of sialic acids, among which N-glycolylneuraminic acid (Neu5Gc) and deaminated neuraminic acid (Kdn) have also been commonly known as major sialic acids other than N-acetylneuraminic acid. Each of these molecular species are known to have a distribution specific to the kind of living organism or biological tissue. In principle, a sialic-acid-containing glycan which contains a sialic acid different from Neu5Ac can also be analyzed similarly to the sialic-acid-containing glycan which contains Neu5Ac. However, in most cases, the abundance of a minor molecular species is considerably lower than that of the most abundant molecular species. Therefore, even when a sialic-acid-linkage specific modification is performed, some of the peaks corresponding to the assumed combination of the linkage types are often undetectable in the mass spectrum, making it difficult to narrow down the composition candidates of the assumed type of sialic-acid-containing glycan.

The present invention has been developed to solve this problem. Its primary objective is to provide a method and device for analyzing a sialic-acid-containing glycan which can improve the efficiency of the task of analyzing a sialic-acid-containing glycan containing various kinds of sialic acids and can also enhance the accuracy of the analysis.

### Solution to Problem

One mode of the method for analyzing a sialic-acid-containing glycan according to the present invention developed for solving the previously described problem is an analysis method for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the method including:

a search condition setting process for setting a glycan search condition;

a peak detection process for detecting, from the mass spectrum data, a representative peak for each isotope peak cluster;

a composition estimation process for estimating, for each representative peak detected in the peak detection process, a glycan composition according to the glycan search condition, and for determining a glycan-composition candidate;

a peak cluster detection process for detecting, from representative peaks detected in the peak detection process, an isomer peak cluster including a plurality of peaks estimated to be identical in the number of sialic acids and in the glycan composition exclusive of the sialic acids;

a glycan composition filtering process for making a selection from the glycan-composition candidates by applying, to each peak included in the isomer peak cluster, predetermined constraint conditions concerning the number of sialic acids contained, the number of linkage types of sialic acids, the kinds of sialic acids, and the identity of the glycan composition exclusive of the sialic acids; and

a display process for creating and displaying a list of the glycan-composition candidates in a manner that enables visual discrimination between the glycan-composition candidates after the selection by the glycan composition filtering process and the other glycan-composition candidates.

One mode of the device for analyzing a sialic-acid-containing glycan according to the present invention developed for solving the previously described problem is an analyzing device for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the device including:

a search condition setter configured to receive a specification of a glycan search condition by a user and set a glycan search condition;

a peak detector configured to detect, from the mass spectrum data, a representative peak for each isotope peak cluster;

a composition estimator configured to estimate, for each representative peak detected by the peak detector, a glycan composition according to the glycan search condition, and to determine a glycan-composition candidate;

a peak cluster detector configured to detect, from representative peaks detected by the peak detector, an isomer peak cluster including a plurality of peaks estimated to be identical in the number of sialic acids and in the glycan composition exclusive of the sialic acids;

a glycan composition filter configured to make a selection from the glycan-composition candidates by applying, to each peak included in the isomer peak cluster, predetermined constraint conditions concerning the number of sialic acids contained, the number of linkage types of sialic acids, the kinds of sialic acids, and the identity of the glycan composition exclusive of the sialic acids; and

a display processor configured to create and display a list of the glycan-composition candidates in a manner that enables visual discrimination between the glycan-composition candidates after the selection by the glycan composition filter and the other glycan-composition candidates.

### Advantageous Effects of Invention

By the previously described modes according to the present invention, even when an ion peak corresponding to a portion of an assumed combination of the linkage types of sialic acids has not been detected for a specific kind of sialic acid among sialic-acid-containing glycans which are identical in the glycan composition exclusive of the sialic acids as well as in the number of sialic acids contained and yet are different in the linkage types of sialic acids and the kinds of sialic acids, the composition candidates of the sialic-acid-containing glycan containing that specific kind of sialic acid can be properly narrowed down and presented to the user. This improves the efficiency of the measurement task, such as an MS/MS analysis for determining whether or not an estimated composition candidate of the sialic-acid-containing glycan is plausible, as well as the efficiency of the task of analyzing the data collected by the measurement, so that the structural analysis of the sialic-acid-containing glycan can be more speedily and accurately performed.

### BRIEF DESCRIPTION OF DRAWINGS

FIG. 1 is a block configuration diagram of one embodiment of a glycan analyzing system including a device for analyzing a sialic-acid-containing glycan according to the present invention.

FIG. 2 is a flowchart showing the procedure of the data-analyzing process in the glycan analyzing system according to the present embodiment.

FIG. 3 is a diagram showing an example of the mass spectrum and illustrating a glycan composition analysis based on the peaks detected in the mass spectrum.

FIG. 4 is a table showing peak clusters detected for the mass spectrum shown in FIG. 3 and the result of the grouping of the peaks into each cluster.

FIG. 5 is one example of the table of the glycan-composition candidates obtained for the six peaks shown in FIG. 4.

FIG. 6 is one example of the table of the glycan-composition candidates obtained for the six peaks shown in FIG. 4.

### DESCRIPTION OF EMBODIMENTS

In the previously described modes of the present invention, a “molecule modified with a sialic-acid-containing glycan” is, for example, a biomolecule, such as a protein, peptide, lipid or nucleic acid, modified with a sialic-acid-containing glycan.

A “modification specific to a sialic-acid linkage type”, on which the previously described modes of the present invention is premised, is typically a SALSA method disclosed in Patent Literature 1 or Non Patent Literature 1 mentioned earlier, but is not limited to this method. It may be any method which causes a sialic-acid-linkage specific chemical modification (derivatization) that allows for the discrimination of two or more different linkage types of sialic acids, such as the α2,3-linkage, α2,6-linkage and α2,8-linkage, by mass difference.

In the previously described modes of the present invention, there is no specific limitation on the type of mass spectrometer for performing a mass spectrometric analysis on a sample containing a sialic-acid-containing glycan (and other compounds). Example of the available mass spectrometers include ion trap mass spectrometers, linear ion trap mass spectrometers, TOF/TOF mass spectrometers, quadrupole time-of-flight (Q-TOF) mass spectrometers, quadrupole ion trap mass spectrometers, and Fourier-transform ion cyclotron resonance mass spectrometers. For the estimation of the glycan composition based on the mass values, a mass spectrometer with a high level of mass accuracy is preferable.

One embodiment of a glycan analyzing system including an analyzing device for carrying out the method for analyzing a sialic-acid-containing glycan according to the present invention is hereinafter described referring to the attached drawings.

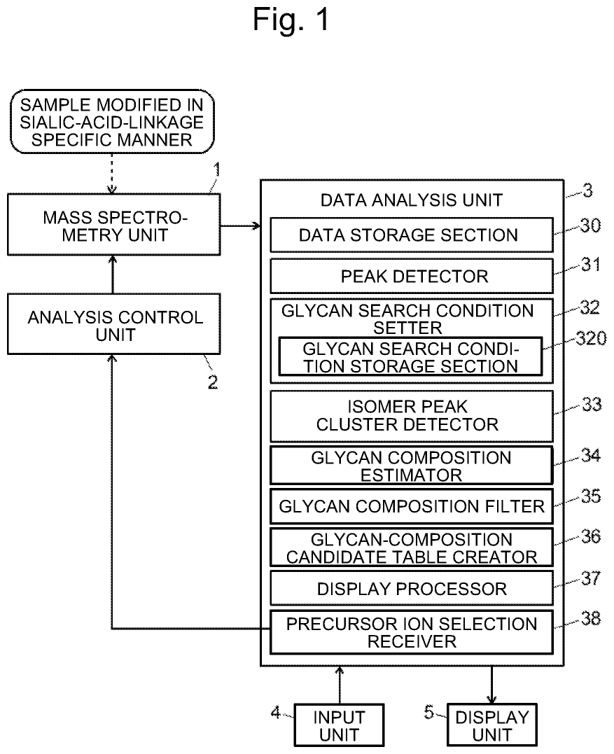

FIG. 1 is a schematic block configuration diagram of the present glycan analyzing system.

As shown in FIG. 1, the present system includes: a mass spectrometry unit 1 configured to perform a mass spectrometric analysis on a sample, an analysis control unit 2 configured to control the mass spectrometry unit 1, a data analysis unit 3 configured to perform an analyzing process on data obtained by a mass spectrometric analysis, as well as an input unit 4 and a display unit 5 serving as a user interface.

The data analysis unit 3 includes, as its functional blocks, a data storage section 30, peak detector 31, glycan search condition setter 32, isomer peak cluster detector 33, glycan composition estimator 34, glycan composition filter 35, glycan-composition candidate table creator 36, display processor 37 and precursor ion selection receiver 38. The glycan search condition setter 32 includes a glycan search condition storage section 320 as a sub-functional block.

There is basically no limitation on the type of mass spectrometry unit 1, although a mass spectrometer having an ion trap, collision cell or similar device capable of fragmenting ions by collision induced dissociation (CID) or other appropriate methods should be used in the case where an MS/MS analysis is carried out, as will be described later. When the composition is to be estimated from mass-to-charge-ratio values as will be described later, the m/z values should preferably be determined with a high level of accuracy and resolving power. Accordingly, a time-of-flight mass separator, Fourier-transform ion cyclotron resonance mass separator or similar device is suitable as the mass separator.

The mass spectrometry unit 1 does not need to be a mass spectrometer in an independent form; a liquid chromatograph mass spectrometer (LC-MS) may also be used. The unit may also be a system in which a plurality of samples are prepared by preparative separation and fractionation of an eluate containing components separated from each other by a liquid chromatograph, and those samples are individually subjected to a mass spectrometric analysis by a mass spectrometer.

The data analysis unit 3 in the present system is actually a personal computer or more sophisticated workstation, on which the functions of the functional blocks shown in FIG. 1 can be realized by executing, on the computer, a dedicated data processing program installed on the same computer. In that case, the input unit 4 is a keyboard and a pointing device (e.g., mouse) provided for the computer, while the display unit 5 is a monitor provided for the same computer.

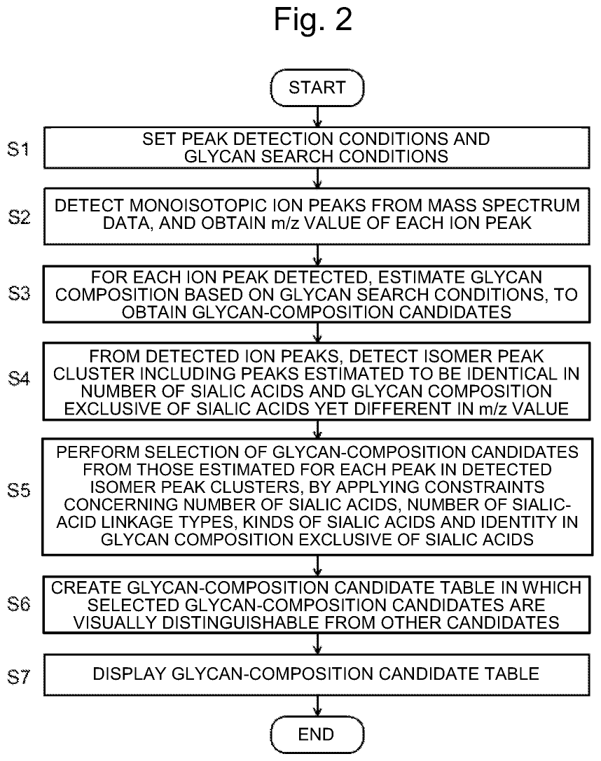

A procedure of the analysis of a sialic-acid-containing glycan in the present glycan analyzing system is hereinafter described, referring to FIGS. 2-6 and including descriptions of an experimental example. FIG. 2 is a flowchart showing the procedure of the glycan analysis mainly carried out by the data analysis unit 3.

For an analysis of a sialic-acid-containing glycan by the present glycan analyzing system, a pretreatment by a sialic-acid-linkage specific chemical modification is initially performed on a sample containing a molecule (such as a glycopeptide or glycolipid) which contains or is modified with a sialic-acid-containing glycan. As for the method for the sialic-acid-linkage specific modification, the SALSA method described in Non Patent Literature 1 (or other related documents) can be used, although the available techniques are not limited to this example. As explained earlier, two molecules which are identical in glycan composition exclusive of the sialic acids will have a mass difference of 28 Da after they have been modified by the SALSA method if a sialic acid contained in the glycan is the α2,3-linkage type in one molecule and the α2,6-linkage type in the other.

Next, a mass spectrometric analysis using the mass spectrometry unit 1 is performed on the sample pretreated by the sialic-acid-linkage specific chemical modification. A set of mass spectrum data covering a predetermined m/z range acquired by the mass spectrometric analysis is sent from the mass spectrometry unit 1 to the data analysis unit 3 and stored in the data storage section 30.

The user specifies the data to be analyzed and issues a command to execute the data analysis through the input unit 4. Then, the data-analyzing process is initiated in the data analysis unit 3 according to the procedure shown in FIG. 2.

The glycan search condition setter 32 displays a glycan search condition setting window on the screen of the display unit 5 and receives entry of glycan search conditions by the user (Step S1). It is also possible to automatically use a default setting of the glycan search conditions, instead of requiring entry by the user. In addition to the glycan search conditions, peak detection conditions for the detection of the peaks from mass spectrum data may also be included in the setting window for the entry by the user. The entered or default settings of the glycan search conditions and peak detection conditions are stored in the glycan search condition storage section 320.

Specifically, the glycan search conditions may include, for example, the sialic-acid-linkage specific modification method to be used, the allowable mass accuracy for the glycan composition estimation, the assumed ion species, as well as the kinds and numbers of sugar residues to be searched for (including sialic acids).

On the other hand, the peak detection conditions may include, for example, the signal intensity or signal-to-noise ratio to be used as the threshold for recognizing a peak.

When a substantive analysis is initiated, the peak detector 31 retrieves the mass spectrum data to be analyzed from the data storage section 30 and detects a monoisotopic ion peak according to the peak detection conditions as the representative peak of each isotope peak cluster. In general, in the case of a molecule derived from a living organism, like glycans, a peak having the smallest m/z value among the plurality of isotopic ion peaks which appear, for example, at intervals of 1 Da can be detected as the monoisotopic ion peak. The peak detector 31 determines the m/z value of each detected ion peak and creates a peak list (Step S2). It is also possible to calculate an average (centroid) of the m/z values of the plurality of isotopic ion peaks in place of the m/z value of the monoisotopic ion peak. In summary, what is required is to determine an m/z value representative of a cluster for each isotopic ion peak cluster originating from the same kind of glycan.

Subsequently, the glycan composition estimator 34 estimates the glycan composition of each peak included in the peak list created in Step S2, according to the glycan search conditions stored in the glycan search condition storage section 320, and determines glycan-composition candidates (Step S3). Specifically, it performs an exhaustive search for the glycan compositions which match the m/z values of the ion peaks within the predetermined allowable mass accuracy under the constraint of the numbers and kinds of sugar residues specified as the glycan search conditions. Each glycan composition which satisfies the search conditions is adopted as a glycan-composition candidate for the ion peak concerned. A successful search for the glycan-composition candidates does not always end with the candidates narrowed down to one; there may be two or more candidates ultimately obtained.

Subsequently, the isomer peak cluster detector 33 detects, from the peaks in the peak list created in Step S2, an isomer peak cluster including a plurality of peaks which are estimated to be identical in the number of sialic acids and in the glycan composition exclusive of the sialic acids (Step S4).

In the present example, the SALSA method is used for the sialic-acid-linkage specific modification. Therefore, according to the SALSA method, each group of ion peaks adjacent to each other at intervals of 28 Da, which equals the difference in m/z between the α2,6-sialic acid and the α2,3-sialic acid, is detected from the peak list. Each of the detected groups of peaks is considered to be a “same-sialic-acid-linked isomer peak cluster” consisting of linkage isomers of the sialic-acid-containing glycans which are identical in the number of sialic acids, in the kinds of sialic acids and in the glycan composition exclusive of the sialic acids. The allowable error of the peak interval used for the detection may be m/z 0.1, for example. The interval of the adjacent peaks considered to be one isomer peak cluster changes depending on the sialic-acid-linkage specific modification method used. Understandably, the number of ion peaks included in one isomer peak cluster changes depending on the number of sialic acids contained in the sialic-acid-containing glycan, linkage types of the sialic acids and other factors.

Furthermore, the isomer peak cluster detector 33 determines whether or not the ion peaks included in a plurality of detected different same-sialic-acid-linked isomer peak clusters are spaced by an m/z-value interval which corresponds to the mass difference of the sugar residues of the different kinds of sialic acids specified in the glycan search conditions. A plurality of isomer peak clusters each of which includes ion peaks corresponding to that m/z-value interval are acknowledged as “different-sialic-acid-linked isomer peak clusters” which are identical in the number of contained sialic acids as well as in the glycan composition exclusive of the sialic acids and are only different in the kind of sialic acid. Furthermore, the ion peaks included in all different-sialic-acid-linked isomer peak clusters which are identical in the number of sialic acids contained as well as in the glycan composition exclusive of the sialic acids are acknowledged as “isomer peak clusters”.

A specific example based on an experiment is as follows: In the experimental example performed by the present inventor, an N-linked glycan which modifies fetuin, which is a kind of protein in the blood of a fetal calf, was cleaved by PNGase, which is a deglycosylation enzyme, and the resulting fragments were enriched to obtain a glycan mixture as a specimen. The sialic acids contained in that specimen were subsequently modified by the SALSA method in a sialic-acid-linkage specific manner, and the reducing terminal of the glycans was labelled with anthranilic acid (AA labelling) to obtain a sample for the analysis. This labelling is a pretreatment for promoting the ionization in a negative ion mode. A mass spectrometric analysis of the sample thus obtained was performed by a matrix assisted laser desorption/ionization ion trap time-of-flight mass spectrometer (MALDI-IT-TOFMS) in a negative ion mode to obtain mass spectrum data.

In the experimental example, the glycan search conditions were set as follows.

Sialic-acid-linkage specific modification method: SALSA method

Labelling method: AA labelling

Allowable mass accuracy for glycan composition estimation: m/z 0.2

Allowable mass-interval error for detection of isomer peak clusters: m/z 0.1

Ion species: deprotonated ion

Kinds and numbers of sugar residues to be searched for: Hexose, 3-15; HexNAc, 2-14; fucose (dHex), 0-2; Neu5Ac (sialic acid), 0-5; and Neu5Gc (sialic acid), 0-5.

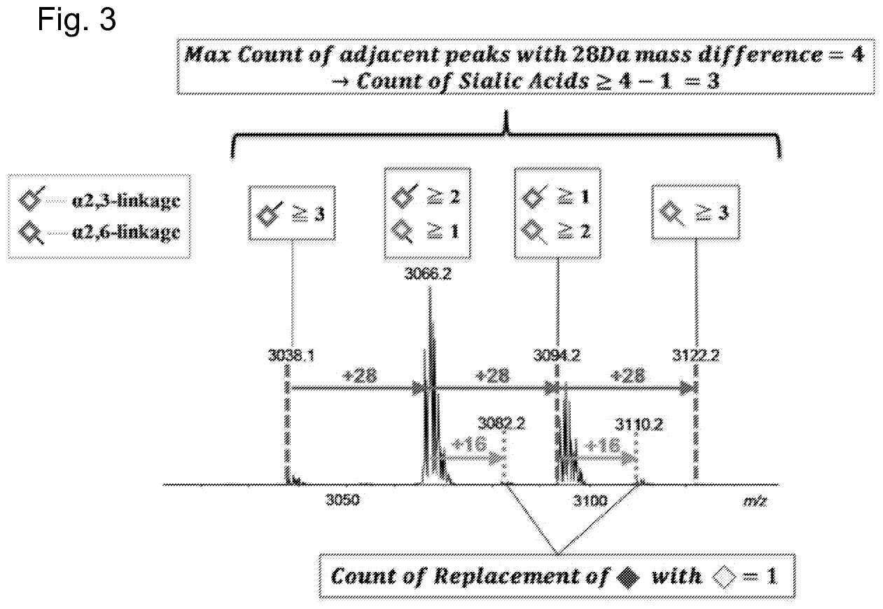

A portion of the mass spectrum obtained by the experimental example is shown in FIG. 3. Within the m/z range shown in FIG. 3, m/z 3038.1, m/z 3066.2, m/z 3082.2, m/z 3094.2, m/z 3110.2 and m/z 3122.2 were detected as monoisotopic ion peaks by the processing of Step S2 performed on the mass spectrum. Based on the peak list including those peaks, the processing of Step S4 was performed. In other words, each group of the peaks spaced at intervals of 28 Da, which is equal to the m/z difference between the α2,3- and α2.6-sialic acids, was detected. Consequently, two same-sialic-acid-linked isomer peak clusters were detected, with one cluster including four singly charged ion peaks located at m/z 3038.1, m/z 3066.2, m/z 3094.2 and m/z 3122.2, and the other cluster including two singly charged ion peaks located at m/z 3082.2 and m/z 3110.2.

It was confirmed that some of the peaks included in each of the two same-sialic-acid-linked isomer peak clusters are adjacent to each other with an interval of 16 Da, which corresponds to the mass difference of the sugar residues of the two kinds of sialic acids specified as glycan search conditions, i.e., N-acetylneuraminic acid (Neu5Ac) and N-glycolylneuraminic acid (Neu5Gc). Therefore, those two same-sialic-acid-linked isomer peak clusters were detected as different-sialic-acid-linked isomer peak clusters, and all peaks included in those different-sialic-acid-linked isomer peak clusters were detected as an isomer peak cluster in which all peaks are identical in the number of sialic acids as well as in the glycan composition exclusive of the sialic acids.

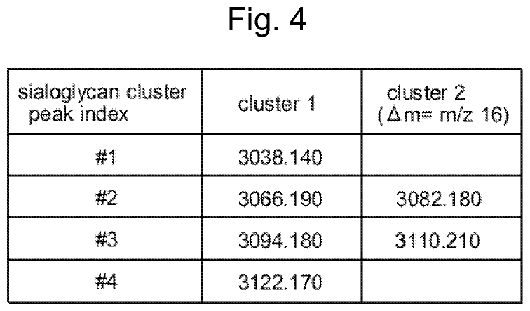

Furthermore, the isomer peak cluster detector 33 classifies a plurality of peaks which are adjacent to each other at intervals equal to the mass difference of the sugar residues of different kinds of sialic acids in the previously described manner, as peaks having the same peak index #n. In the previously described experimental example, as shown in FIG. 4, the peak having the smallest mass value of m/z 3038.1 among all peaks included in the two different-sialic-acid-linked isomer peak clusters (“cluster 1” and “cluster 2”) was classified as a peak with peak index #1 and included in the same-sialic-acid-linked isomer peak cluster 1 (“cluster 1”). The peak having a mass value of m/z 3066.2, which was the second smallest mass value among the peak clusters and 28 Da larger than the mass value of the peak with peak index #1, was classified as peak index #2. A peak detected at m/z 3082.18 with a mass difference of 16 Da from the peak at m/z 3066.2 was classified as the same peak index #2 and included in the same-sialic-acid-linked isomer peak cluster 2 (“cluster 2”). Similarly, the peaks at m/z 3094.2 and m/z 3110.2 were classified as peak index #3, and the peak at m/z 3122.2 was classified as peak index #4.

In the present example, the different-sialic-acid-linked isomer peak clusters were detected from a plurality of peaks included in the two same-sialic-acid-linked isomer peak clusters. As another possibility, a different-sialic-acid-linked isomer peak cluster may be formed for a portion of the peaks in one same-sialic-acid-linked isomer peak cluster, by detecting, for each peak, one different-sialic-acid-containing glycan peak having a mass difference corresponding to the mass difference of the glycan residues of the different kinds of sialic acids concerned.

The description returns to the flowchart of FIG. 2 and further continues.

Next, for each of the glycan-composition candidates obtained in Step S3 for each peak included in the isomer peak clusters, the glycan composition filter 35 makes a selection of glycan-composition candidates by applying constraint conditions concerning the number of sialic acids, number of linkage types of sialic acids, kinds of sialic acids, and identity of the glycan composition exclusive of the sialic acids, as will be hereinafter illustrated (Step S5).

### Constraint Conditions

(1) In the present example, four peak indices have been obtained by the isomer peak cluster detector 33. Accordingly, for all of those peaks, glycan composition candidates which contain three or more same number of sialic acids and are identical in composition exclusive of the sialic acids are plausible as sialic-acid-containing glycans. In general, when N peak indices have been obtained, the glycans corresponding to the original peaks contain N-1 or more sialic acids.

(2) Additionally, from a comparison of the masses of those peaks, it is possible to consider that each peak classified as peak index #1 should contain three or more α2,3-linked sialic acids, each peak classified as peak index #2 should contain two or more α2,3-linked sialic acids and one or more α2,6-linked sialic acids, each peak classified as peak index #3 should contain one or more α2,3-linked sialic acids and two or more α2,6-linked sialic acids, and each peak classified as peak index #4 should contain three or more α2,6-linked sialic acids. Each peak classified into the same-sialic-acid-linked isomer peak cluster 2 should have a glycan composition which results from the replacement of one kind of sialic acid, from Neu5Ac to Neu5Gc, in the glycan composition of the peak having the same peak index in the same-sialic-acid-linked isomer peak cluster 1.

It should be noted that the peaks shown in FIG. 3 are accompanied by descriptions of the aforementioned constraint conditions.

In normal cases, the number of glycan-composition candidates are considerably decreased as a result of the selection by the glycan composition filter 35. The glycan-composition candidate table creator 36 collects glycan-composition candidates estimated in Step S3 for each peak included in the isomer peak clusters detected in Step S4, and displays a glycan-composition candidate table in which the collected candidates are related to each peak in the isomer peak clusters. Furthermore, the glycan-composition candidate table creator 36 shows the glycan-composition candidates in this glycan-composition candidate table in a manner that enables visual discrimination between the candidates which have been selected in Step S5 (and remain after the filtering) and those which have not been selected (Step S6).

The display processor 37 displays the created glycan-composition candidate table on the screen of the display unit 5 (Step S7).

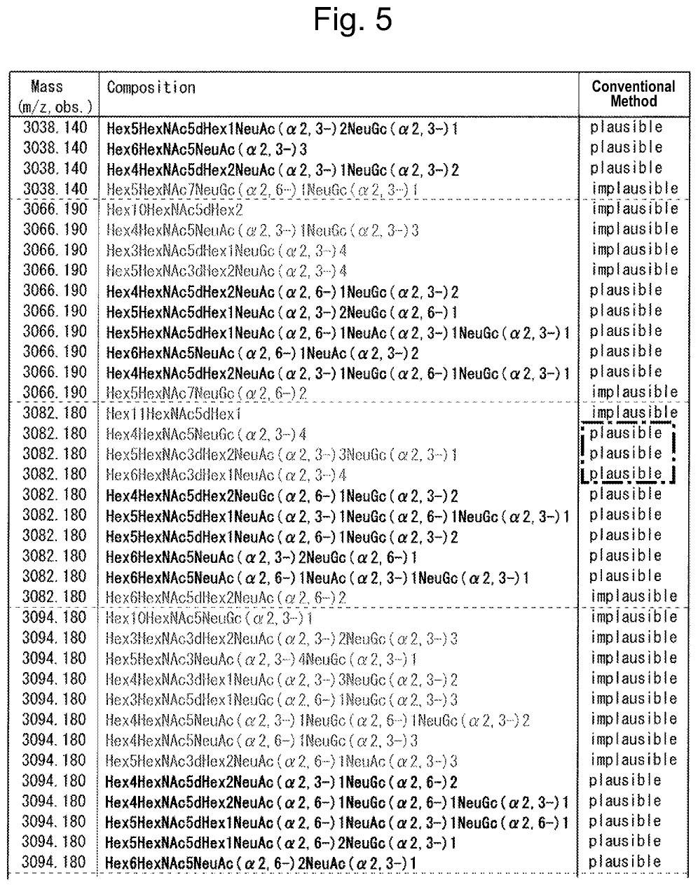

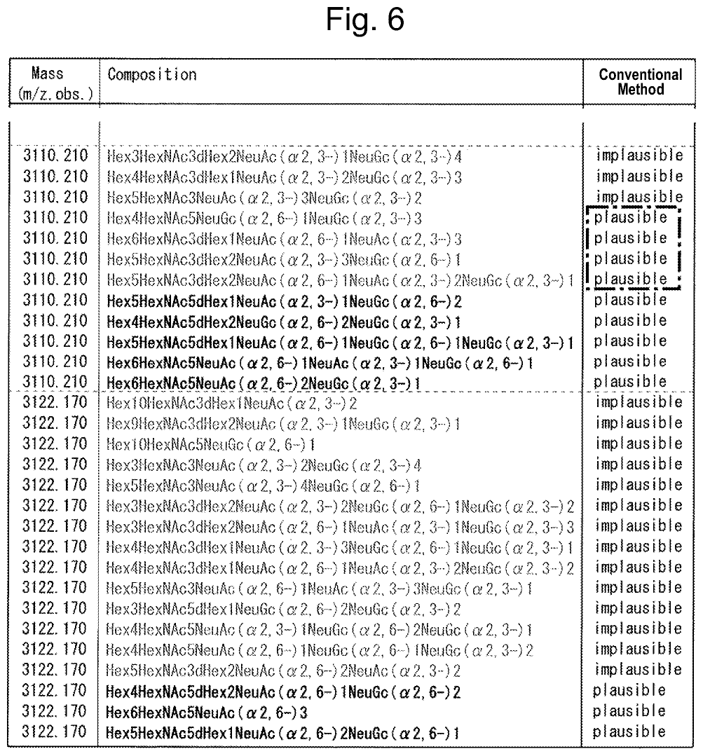

FIGS. 5 and 6 show an example of the table showing the glycan-composition candidates obtained for six peaks in the aforementioned experimental example. In this example, the glycan-composition candidates (“Composition”) selected as plausible by the glycan composition filter 35 are shown in bold font, while the other glycan-composition candidates are shown in fine and pale letters. The manner of the discrimination on the display is not limited to this one; for example, the two kinds of glycan-composition candidates may be shown in different letter colors, with different background colors or in any other appropriate manner that prevents visual mistaking. It is also possible to selectively display only the plausible or implausible glycan-composition candidates according to an instruction by the user.

The “Conventional Method” field at the right end of FIGS. 5 and 6 shows the discrimination between the glycan-composition candidates considered to be “plausible” and those considered to be “implausible” by the technique described in Patent Literature 1. It should be noted that this field has been added for a comparison between the technique according to the present invention and the conventional technique. This field will not be included in the glycan-composition candidate table to be displayed in the device according to the present embodiment.

A comparison between the discrimination by bold/fine letters in the “Composition” field and the discrimination by “plausible/implausible” in the “Conventional Method” field in FIGS. 5 and 6 shows that some of the glycan-composition candidates which have been selected as “plausible” candidates by the conventional method are considered to be implausible by the method according to the present embodiment.

Specifically, a disagreement between the conventional technique and the method according to the present embodiment is present in the estimation results of the plausibility of the glycan-composition candidates for two peaks at m/z 3082.18 and m/z 3110.21 belonging to “cluster 2”; the glycan-composition candidates surrounded by the chain-line frames in the “Conventional Method” field in FIGS. 5 and 6 were considered to be “plausible” by the conventional method, whereas those candidates are considered to be implausible and excluded from the selection by the technique according to the present embodiment.

As for the glycans belonging to “cluster 1”, the estimated glycan-composition candidates are appropriately narrowed down by the conventional method. In this point, the conventional method is satisfactorily useful. By comparison, in the method according to the present embodiment, since unified constraint conditions are used among the sialic-acid-containing glycan clusters each of which contains different kinds of sialic acids, the glycan compositions can be appropriately narrowed down even when only a portion of the linkage isomer peaks of a glycan containing a certain kind of sialic acid have been detected. Therefore, an advantage over the conventional method exists in that the process of narrowing down the candidates can be performed for a wider range of glycans.

When the user wants to experimentally determine the glycan composition or glycan structure of a peak for which a plurality of glycan-composition candidates have been nominated, it is necessary to perform an MS/MS analysis in which an ion corresponding to that peak is selected as the target. In that case, the user performs an operation for indicating a desired peak, for example, in the displayed mass spectrum or glycan-composition candidate table. Then, the precursor ion selection receiver 38 responds to this operation and selects the indicated ion peak as the precursor ion for the MS/MS analysis.

The information of this selection is sent to the analysis control unit 2. The analysis control unit 2 operates the mass spectrometry unit 1 so as to perform an MS/MS analysis, or more specifically, a product ion scan measurement employing an ion dissociation technique, such as collision induced dissociation, with the selected precursor ion as the target. The mass spectrometry unit 1 performs an MS/MS analysis on a sample containing glycans which have undergone the sialic-acid-linkage specific modification, to obtain MS/MS spectrum data. In the MS/MS spectrum, a plurality of product ion peaks originating from the targeted sialic-acid-containing glycan are observed. Based on the m/z values of those peaks, the user can determine which of the plurality of glycan-composition candidates is the most plausible candidate, or whether or not the single glycan-composition candidate is an appropriate candidate.

As described thus far, a structural analysis of sialic-acid-containing glycans, including the linkage type of sialic acids, can be efficiently performed by the glycan analyzing system according to the present embodiment.

The order of the steps in the flowchart shown in FIG. 2 can be appropriately changed as long as it does not affect the essential content of the processing in each step. For example, Steps S3 and S4 can be transposed. The setting of the glycan search conditions in Step S1 can be performed at any time before the glycan composition is estimated in Step S3.

The isomer peak cluster detector 33 may be configured to detect different-sialic-acid-linked isomer peak clusters for all possible combinations of the selectable sialic acids, regardless of whether or not a plurality of kinds of sialic acids are specified as the sugar residues to be searched for in the glycan search condition setter 32, and to perform the glycan composition estimation after adding, to the sugar residues to be searched for in the glycan search condition setter 32, the combination of the kinds of sialic acids corresponding to the mass difference of the peaks included in the detected clusters.

The previous embodiment is a mere example of the present invention. Any change, modification, addition or the like appropriately made within the gist of the present invention will naturally be included within the scope of claims of the present application.

### Various Modes

A person skilled in the art can understand that the previously described illustrative embodiment is a specific example of the following modes of the present invention.

(Clause 1) One mode of the method for analyzing a sialic-acid-containing glycan the present invention is an analysis method for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the method including:

a search condition setting process for setting a glycan search condition;

a peak detection process for detecting, from the mass spectrum data, a representative peak for each isotope peak cluster;

a composition estimation process for estimating, for each representative peak detected in the peak detection process, a glycan composition according to the glycan search condition, and for determining a glycan-composition candidate;

a peak cluster detection process for detecting, from representative peaks detected in the peak detection process, an isomer peak cluster including a plurality of peaks estimated to be identical in the number of sialic acids and in the glycan composition exclusive of the sialic acids;

a glycan composition filtering process for making a selection from the glycan-composition candidates by applying, to each peak included in the isomer peak cluster, predetermined constraint conditions concerning the number of sialic acids contained, the number of linkage types of sialic acids, the kinds of sialic acids, and the identity of the glycan composition exclusive of the sialic acids; and

a display process for creating and displaying a list of the glycan-composition candidates in a manner that enables visual discrimination between the glycan-composition candidates after the selection by the glycan composition filtering process and the other glycan-composition candidates.

(Clause 3) One mode of the device for analyzing a sialic-acid-containing glycan the present invention is an analyzing device for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the device including:

a search condition setter configured to receive a specification of a glycan search condition by a user and set a glycan search condition;

a peak detector configured to detect, from the mass spectrum data, a representative peak for each isotope peak cluster;

a composition estimator configured to estimate, for each representative peak detected by the peak detector, a glycan composition according to the glycan search condition, and to determine a glycan-composition candidate;

a peak cluster detector configured to detect, from representative peaks detected by the peak detector, an isomer peak cluster including a plurality of peaks estimated to be identical in the number of sialic acids and in the glycan composition exclusive of the sialic acids;

a glycan composition filter configured to make a selection from the glycan-composition candidates by applying, to each peak included in the isomer peak cluster, predetermined constraint conditions concerning the number of sialic acids contained, the number of linkage types of sialic acids, the kinds of sialic acids, and the identity of the glycan composition exclusive of the sialic acids; and

a display processor configured to create and display a list of the glycan-composition candidates in a manner that enables visual discrimination between the glycan-composition candidates after the selection by the glycan composition filter and the other glycan-composition candidates.

In the method for analyzing a sialic-acid-containing glycan according to Clause 1, the processes are not always carried out in the aforementioned order; the order of execution of those processes may be appropriately changed. For example, the peak cluster detection process may be carried out earlier than the composition estimation process. In that case, the estimation of the composition only needs to be selectively performed for the peaks included in isomer peak clusters among the peaks detected in the peak detection process. This does not cause any problem in the subsequent processing.

By using the method for analyzing a sialic-acid-containing glycan according to Clause 1 and the device for analyzing a sialic-acid-containing glycan according to Clause 3, even when an ion peak corresponding to a portion of an assumed combination of the linkage types of sialic acids has not been detected for a specific kind of sialic acid among sialic-acid-containing glycans which are identical in the glycan composition exclusive of the sialic acids as well as in the number of sialic acids contained and yet are different in the linkage types of sialic acids and the kinds of sialic acids, the composition candidates of the sialic-acid-containing glycan containing that specific kind of sialic acid can be properly narrowed down and presented to the user. This improves the efficiency of the measurement task, such as an MS/MS analysis for determining whether or not an estimated composition candidate of the sialic-acid-containing glycan is plausible, as well as the efficiency of the task of analyzing the data collected by the measurement, so that the structural analysis of the sialic-acid-containing glycan can be more speedily and accurately performed.

(Clause 2) In the method for analyzing a sialic-acid-containing glycan according to Clause 1, the peak cluster detection process may include additionally attempting a detection of an isomer peak cluster for a kind of sialic acid which is not specified beforehand, and once more performing the process of detecting an isomer peak cluster after adding the kind of sialic acid which is not specified beforehand to the target of the search in the glycan search condition, based on isomer peak cluster information obtained as a result of the attempted detection.

By the method for analyzing a sialic-acid-containing glycan according to Clause 2, even when a sialic-acid-containing glycan which contains a kind of sialic acid unexpected by the user was contained in the sample, the structural analysis of that sialic-acid-containing glycan can be performed.

### REFERENCE SIGNS LIST

### 1 . . . Mass Spectrometry Unit

### 2 . . . Analysis Control Unit

### 3 . . . Data Analysis Unit

### 30 . . . Data Storage Section

### 31 . . . Peak Detector

### 32 . . . Glycan Search Condition Setter

### 320 . . . Glycan Search Condition Storage Section

### 33 . . . Isomer Peak Cluster Detector

### 34 . . . Glycan Composition Estimator

### 35 . . . Glycan Composition Filter

### 36 . . . Glycan-Composition Candidate Table Creator

### 37 . . . Display Processor

### 38 . . . Precursor Ion Selection Receiver

### 4 . . . Input Unit

### 5 . . . Display Unit

1. A method for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the method comprising:
a search condition setting process for setting a glycan search condition;
a peak detection process for detecting, from the mass spectrum data, a representative peak for each isotope peak cluster;
a composition estimation process for estimating, for each representative peak detected in the peak detection process, a glycan composition according to the glycan search condition, and for determining a glycan-composition candidate;
a peak cluster detection process for detecting, from representative peaks detected in the peak detection process, an isomer peak cluster including a plurality of peaks estimated to be identical in a number of sialic acids and in a glycan composition exclusive of the sialic acids;
a glycan composition filtering process for making a selection from the glycan-composition candidates by applying, to each peak included in the isomer peak cluster, predetermined constraint conditions concerning the number of sialic acids contained, a number of linkage types of sialic acids, kinds of sialic acids, and an identity of the glycan composition exclusive of the sialic acids; and
a display process for creating and displaying a list of the glycan-composition candidates in a manner that enables visual discrimination between the glycan-composition candidates after the selection by the glycan composition filtering process and other glycan-composition candidates.

2. The method for analyzing a sample containing a sialic-acid-containing glycan according to claim 1, wherein the peak cluster detection process includes additionally attempting a detection of an isomer peak cluster for a kind of sialic acid which is not specified beforehand, and once more performing a process of detecting an isomer peak cluster after adding the kind of sialic acid which is not specified beforehand to a target of the search in the glycan search condition, based on isomer peak cluster information obtained as a result of the attempted detection.

3. A device for analyzing a sample containing a sialic-acid-containing glycan including a modification specific to a sialic-acid linkage type, or a sample containing a molecule modified with the same glycan, based on mass spectrum data obtained by a mass spectrometric analysis of the sample, the device comprising:
a search condition setter configured to receive a specification of a glycan search condition by a user and set a glycan search condition;
a peak detector configured to detect, from the mass spectrum data, a representative peak for each isotope peak cluster;
a composition estimator configured to estimate, for each representative peak detected by the peak detector, a glycan composition according to the glycan search condition, and to determine a glycan-composition candidate;
a peak cluster detector configured to detect, from representative peaks detected by the peak detector, an isomer peak cluster including a plurality of peaks estimated to be identical in a number of sialic acids and in a glycan composition exclusive of the sialic acids;
a glycan composition filter configured to make a selection from the glycan-composition candidates by applying, to each peak included in the isomer peak cluster, predetermined constraint conditions concerning the number of sialic acids contained, a number of linkage types of sialic acids, kinds of sialic acids, and an identity of the glycan composition exclusive of the sialic acids; and
a display processor configured to create and display a list of the glycan-composition candidates in a manner that enables visual discrimination between the glycan-composition candidates after the selection by the glycan composition filter and other glycan-composition candidates.
